(19) United States
(12) Patent Application Publication
(10) Pub. No.: US 2015/0097935 A1
(43) Pub. Date: Apr. 9, 2015

(54) INTEGRATED TRACKING WITH WORLD MODELING

(71) Applicant: United Sciences, LLC, Atlanta, GA (US)

(72) Inventors: Karol Hatzilias, Atlanta, GA (US); Harris Bergman, Marietta, GA (US); Rob Blenis, Atlanta, GA (US); Wess Eric Sharpe, Atlanta, GA (US)

(21) Appl. No.: 14/049,687

(22) Filed: Oct. 9, 2013

(51) Int. Cl. G06T 7/00; A61B 1/045; A61B 1/227

(52) U.S. Cl. CPC G06T 7/0044; A61B 1/227; A61B 1/045; G06T 2207/30244; G06T 2207/30004; G06T 2207/10068
USPC 348/65

(57) ABSTRACT

Disclosed are various embodiments for determining a pose of a mobile device by analyzing a digital image captured by at least one imaging device to identify a plurality of regions in a fiducial marker indicative of a pose of the mobile device. A fiducial marker may comprise a circle-of-dots pattern, the circle-of-dots pattern comprising an arrangement of dots of varied sizes. The pose of the mobile device may be used to generate a three-dimensional reconstruction of an item subject to a scan via the mobile device.

### CROSS REFERENCE TO RELATED APPLICATIONS

This application is related to U.S. patent application Ser. No. ______, filed on Oct. ______, 2013 (Attorney Docket No. 52105-1010) and entitled “Tubular Light Guide,” U.S. patent application Ser. No. ______, filed on Oct. ______, 2013 (Attorney Docket No. 52105-1020) and entitled “Tapered Optical Guide,” U.S. patent application Ser. No. ______, filed on Oct. ______, 2013 (Attorney Docket No. 52105-1030) and entitled “Display for Three-Dimensional Imaging,” U.S. patent application Ser. No. ______, filed on Oct. ______, 2013 (Attorney Docket No. 52105-1040) and entitled “Fan Light Element,” U.S. patent application Ser. No. ______, filed on Oct. ______, 2013 (Attorney Docket No. 52105-1060) and entitled “Integrated Tracking with Fiducial-based Modeling,” U.S. patent application Ser. No. ______, filed on Oct. ______, 2013 (Attorney Docket No. 52105-1070) and entitled “Integrated Calibration Cradle,” and U.S. patent application Ser. No. ______, filed on Oct. ______, 2013 (Attorney Docket No. 52105-1080) and entitled “Calibration of 3D Scanning Device,” all of which are hereby incorporated by reference in their entirety.

### BACKGROUND

There are various needs for understanding the shape and size of cavity surfaces, such as body cavities. For example, hearing aids, hearing protection, custom head phones, and wearable computing devices may require impressions of a patient's ear canal. To construct an impression of an ear canal, audiologists may inject a silicone material into a patient's ear canal, wait for the material to harden, and then provide the mold to manufacturers who use the resulting silicone impression to create a custom fitting in-ear device. As may be appreciated, the process is slow, expensive, and unpleasant for the patient as well as a medical professional performing the procedure.

Computer vision and photogrammetry generally relates to acquiring and analyzing images in order to produce data by electronically understanding an image using various algorithmic methods. For example, computer vision may be employed in event detection, object recognition, motion estimation, and various other tasks.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1A-1C are drawings of an otoscanner according to various embodiments of the present disclosure.

FIG. 2 is a drawing of the otoscanner of FIGS. 1A-1C performing a scan of a surface according to various embodiments of the present disclosure.

FIG. 3 is a pictorial diagram of an example user interface rendered by a display in data communication with the otoscanner of FIGS. 1A-1C according to various embodiments of the present disclosure.

FIG. 4 is a drawing of a fiducial marker that may be used by the otoscanner of FIGS. 1A-1C in pose estimation according to various embodiments of the present disclosure.

FIG. 5 is a drawing of the otoscanner of FIGS. 1A-1C conducting a scan of an ear encompassed by the fiducial marker of FIG. 4 that may be used in pose estimation according to various embodiments of the present disclosure.

FIG. 6 is a drawing of a camera model that may be employed in an estimation of a pose of the scanning device of FIGS. 1A-1C according to various embodiments of the present disclosure.

FIG. 7 is a drawing of a partial bottom view of the otoscanner of FIGS. 1A-1C according to various embodiments of the present disclosure.

FIG. 8 is a drawing illustrating the epipolar geometric relationships of at least two imaging devices in data communication with the otoscanner of FIGS. 1A-1C according to various embodiments of the present disclosure.

FIG. 9 is a flowchart illustrating one example of functionality implemented as portions of a pose estimate application executed in the otoscanner of FIGS. 1A-1C according to various embodiments of the present disclosure.

FIG. 10 is a schematic block diagram that provides one example illustration of a computing environment employed in the otoscanner of FIGS. 1A-1C according to various embodiments of the present disclosure.

### DETAILED DESCRIPTION

The present disclosure relates to a mobile scanning device configured to scan and generate images and reconstructions of surfaces. Advancements in computer vision permit imaging devices, such as conventional cameras, to be employed as sensors useful in determining locations, shapes, and appearances of objects in a three-dimensional space. For example, a position and an orientation of an object in a three-dimensional space may be determined relative to a certain world coordinate system utilizing digital images captured via image capturing devices. As may be appreciated, the position and orientation of the object in the three-dimensional space may be beneficial in generating additional data about the object, or about other objects, in the same three-dimensional space.

For example, scanning devices may be used in various industries to scan objects to generate data pertaining to the objects being scanned. A scanning device may employ an imaging device, such as a camera, to determine information about the object being scanned, such as the size, shape, or structure of the object, the distance of the object from the scanning device, etc.

As a non-limiting example, a scanning device may include an otoscanner configured to visually inspect or scan the ear canal of a human or animal. An otoscanner may comprise one or more cameras that may be beneficial in generating data about the ear canal subject of the scan, such as the size, shape, or structure of the ear canal. This data may be used in generating three-dimensional reconstructions of the ear canal that may be useful in customizing in-ear devices, for example but not limited to, hearing aids or wearable computing devices.

Determining the size, shape, or structure of an object subject to a scan, may require information about a position of the object relative to the scanning device conducting the scan. For example, during a scan, a distance of an otoscanner from an ear canal may be beneficial in determining the shape of the ear canal. An estimated position of the scanning device relative to the object being scanned (i.e., the pose estimate) may be generated using various methods, as will be described in greater detail below.

According to one embodiment, determining an accurate pose estimate for a scanning device (e.g., an otoscanner) may comprise employing one or more fiducial markers to be imaged via one or more imaging devices in data communication with the scanning device. By being imaged via the imaging devices, the fiducial marker may act as a point of reference or as a measure in estimating a pose (or position) of the scanning device. A fiducial marker may comprise, for example, a circle-of-dots fiducial marker comprising a plurality of machine-identifiable regions (also known as “blobs”), as will be described in greater detail below. In other embodiments, the tracking targets may be naturally occurring features surrounding and/or within the cavity to be scanned.

As a scanning device is performing a scan of an object, the one or more imaging devices may generate one or more digital images. The digital images may be analyzed for the presence of at least a portion of the one or more circle-of-dots fiducial markers. Subsequently, an identified portion of the one or more circle-of-dots fiducial markers may be analyzed and used in determining a relatively accurate pose estimate for the scanning device. The pose estimate may be used in generating three-dimensional reconstructions of an ear canal, as will be described in greater detail below.

In the following discussion, a general description of the system and its components is provided, followed by a discussion of the operation of the same.

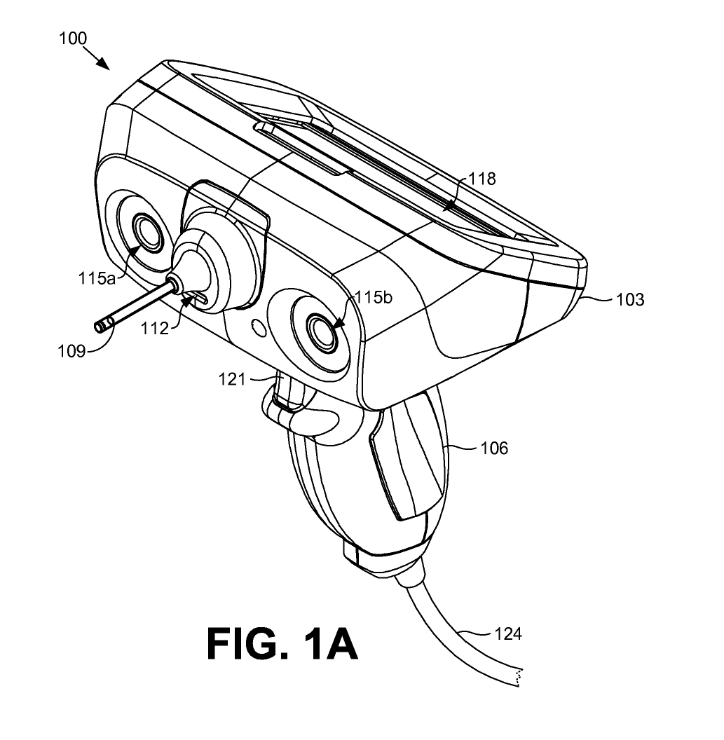

With reference to FIG. 1A, shown is an example drawing of a scanning device 100 according to various embodiments of the present disclosure. The scanning device 100, as illustrated in FIG. 1A, may comprise, for example, a body 103 and a hand grip 106. Mounted upon the body 103 of the scanning device 100 are a probe 109, a fan light element 112, and a plurality of tracking sensors comprising, for example, a first imaging device 115a and a second imaging device 115b. According to various embodiments, the scanning device 100 may further comprise a display screen 118 configured to render images captured via the probe 109, the first imaging device 115a, the second imaging device 115b, and/or other imaging devices.

The hand grip 106 may be configured such that the length is long enough to accommodate large hands and the diameter is small enough to provide enough comfort for smaller hands. A trigger 121, located within the hand grip 106, may perform various functions such as initiating a scan of a surface, controlling a user interface rendered in the display, and/or otherwise modifying the function of the scanning device 100.

The scanning device 100 may further comprise a cord 124 that may be employed to communicate data signals to external computing devices and/or to power the scanning device 100. As may be appreciated, the cord 124 may be detachably attached to facilitate the mobility of the scanning device 100 when held in a hand via the hand grip 106. According to various embodiments of the present disclosure, the scanning device 100 may not comprise a cord 124, thus acting as a wireless and mobile device capable of wireless communication.

The probe 109 mounted onto the scanning device 100 may be configured to guide light received at a proximal end of the probe 109 to a distal end of the probe 109 and may be employed in the scanning of a surface cavity, such as an ear canal, by placing the probe 109 near or within the surface cavity. During a scan, the probe 109 may be configured to project a 360-degree ring onto the cavity surface and capture reflections from the projected ring to reconstruct the image, size, and shape of the cavity surface. In addition, the scanning device 100 may be configured to capture video images of the cavity surface by projecting video illuminating light onto the cavity surface and capturing video images of the cavity surface.

The fan light element 112 mounted onto the scanning device 100 may be configured to emit light in a fan line for scanning an outer surface. The fan light element 112 comprises a fan light source projecting light onto a single element lens to collimate the light and generate a fan line for scanning the outer surface. By using triangulation of the reflections captured when projected onto a surface, the imaging sensor within the scanning device 100 may reconstruct the scanned surface.

FIG. 1A illustrates an example of a first imaging device 115a and a second imaging device 115b mounted on or within the body 103 of the scanning device 100, for example, in an orientation that is opposite from the display screen 118. The display screen 118, as will be discussed in further detail below, may be configured to render digital media of a surface cavity captured by the scanning device 100 as the probe 109 is moved within the cavity. The display screen 118 may also display, either separately or simultaneously, real-time constructions of three-dimensional images corresponding to the scanned cavity, as will be discussed in greater detail below.

Referring next to FIG. 1B, shown is another drawing of the scanning device 100 according to various embodiments. In this example, the scanning device 100 comprises a body 103, a probe 109, a hand grip 106, a fan light element 112, a trigger 121, and a cord 124 (optional), all implemented in a fashion similar to that of the scanning device described above with reference to FIG. 1A. In the examples of FIGS. 1A and 1B, the scanning device 100 is implemented with the first imaging device 115a and the second imaging device 115b mounted within the body 103 without hindering or impeding a view of the first imaging device 115a and/or a second imaging device 115b. According to various embodiments of the present disclosure, the placement of the imaging devices 115 may vary as needed to facilitate accurate pose estimation, as will be discussed in greater detail below.

Turning now to FIG. 1C, shown is another drawing of the scanning device 100 according to various embodiments. In the non-limiting example of FIG. 1C, the scanning device 100 comprises a body 103, a probe 109, a hand grip 106, a trigger 121, and a cord 124 (optional), all implemented in a fashion similar to that of the scanning device described above with reference to FIGS. 1A-1B.

In the examples of FIGS. 1A, 1B, and 1C, the scanning device 100 is implemented with the probe 109 mounted on the body 103 between the hand grip 106 and the display screen 118. The display screen 118 is mounted on the opposite side of the body 103 from the probe 109 and distally from the hand grip 106. To this end, when an operator takes the hand grip 106 in the operator's hand and positions the probe 109 to scan a surface, both the probe 109 and the display screen 118 are easily visible at all times to the operator.

Further, the display screen 118 is coupled for data communication to the imaging devices 115 (not shown). The display screen 118 may be configured to display and/or render images of the scanned surface. The displayed images may include digital images or video of the cavity captured by the probe 109 and the fan light element 112 (not shown) as the probe 109 is moved within the cavity. The displayed images may also include real-time constructions of three-dimensional images corresponding to the scanned cavity. The display screen 118 may be configured, either separately or simultaneously, to display the video images and the three-dimensional images, as will be discussed in greater detail below.

According to various embodiments of the present disclosure, the imaging devices 115 of FIGS. 1A, 1B, and 1C, may comprise a variety of cameras to capture one or more digital images of a surface cavity subject to a scan. A camera is described herein as a ray-based sensing device and may comprise, for example, a charge-coupled device (CCD) camera, a complementary metal-oxide semiconductor (CMOS) camera, or any other appropriate camera. Similarly, the camera employed as an imaging device 115 may comprise one of a variety of lenses such as: apochromat (APO), process with pincushion distortion, process with barrel distortion, fisheye, stereoscopic, soft-focus, infrared, ultraviolet, swivel, shift, wide angle, any combination thereof, and/or any other appropriate type of lens.

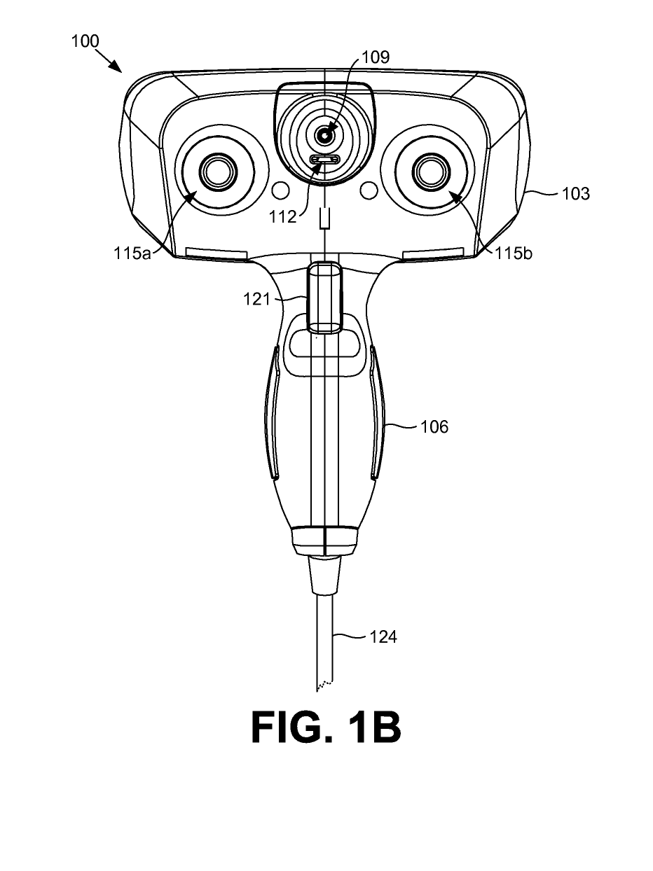

Moving on to FIG. 2, shown is an example of the scanning device 100 emitting a fan line 203 for scanning a surface. In this example, the scanning device 100 is scanning the surface of an ear 206. However, it should be noted that the scanning device 100 may be configured to scan other types of surfaces and is not limited to human or animal applications. The fan light element 112 may be designed to emit a fan line 203 formed by projecting divergent light generated by the fan light source onto the fan lens. As the fan line 203 is projected onto a surface, the lens system may capture reflections of the fan line 203. An image sensor may use triangulation to construct an image of the scanned surface based at least in part on the reflections captured by the lens system. Accordingly, the constructed image may be displayed on the display screen 118 (FIGS. 1A and 1C) and/or other displays in data communication with the scanning device 100.

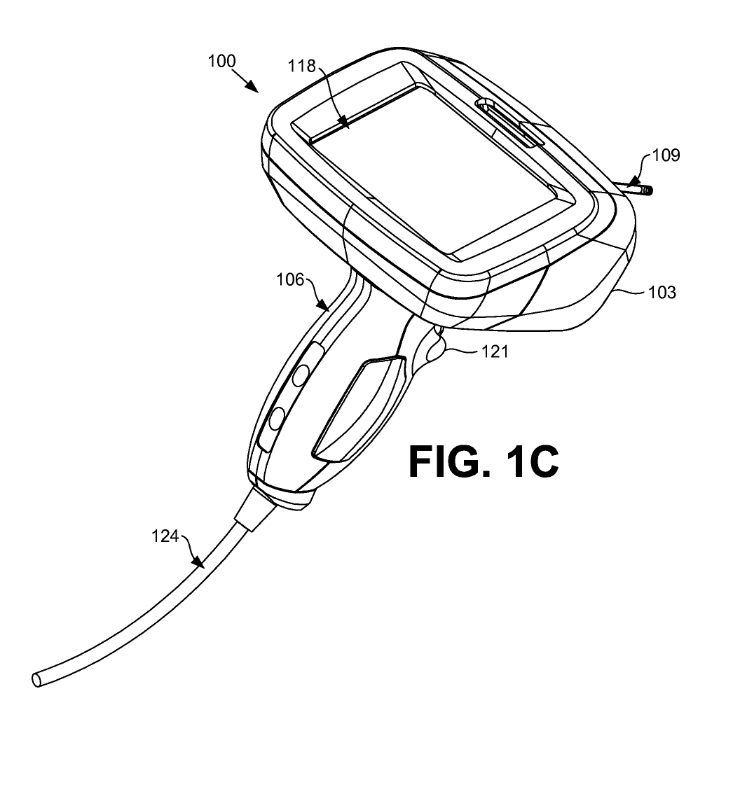

Referring next to FIG. 3, shown is an example user interface that may be rendered, for example, on a display screen 118 within the scanning device 100 or in any other display in data communication with the scanning device 100. In the non-limiting example of FIG. 3, a user interface may comprise a first portion 303a and a second portion 303b rendered separately or simultaneously in a display. For example, in the first portion 303a, a real-time video stream may be rendered, providing an operator of the scanning device 100 with a view of a surface cavity being scanned. The real-time video stream may be generated via the probe 109 or via one of the imaging devices 115.

In the second portion 303b, a real-time three-dimensional reconstruction of the object being scanned may be rendered, providing the operator of the scanning device 100 with an estimate regarding what portion of the surface cavity has been scanned. For example, the three-dimensional reconstruction may be non-existent as a scan of a surface cavity is initiated by the operator. As the operator progresses in conducting a scan of the surface cavity, a three-dimensional reconstruction of the surface cavity may be generated portion-by-portion, progressing into a complete reconstruction of the surface cavity at the completion of the scan. In the non-limiting example of FIG. 3, the first portion 303a may comprise, for example, an inner view of an ear canal 306 generated by the probe 109 and the second portion 303b may comprise, for example, a three-dimensional reconstruction of an ear canal 309, or vice versa.

A three-dimensional reconstruction of an ear canal 309 may be generated via one or more processors internal to the scanning device 100, external to the scanning device 100, or a combination thereof. Generating the three-dimensional reconstruction of the object subject to the scan may require information related to the pose of the scanning device 100. The three-dimensional reconstruction of the ear canal 309 may further comprise, for example, a probe model 310 emulating a position of the probe 109 relative to the surface cavity being scanned by the scanning device. Determining the information that may be used in the three-dimensional reconstruction of the object subject to the scan and the probe model 310 will be discussed in greater detail below.

A notification area 312 may provide the operator of the scanning device with notifications, whether assisting the operator with conducting a scan or warning the operator of potential harm to the object being scanned. Measurements 315 may be rendered in the display to assist the operator in conducting scans of surface cavities at certain distances and/or depths. A bar 318 may provide the operator with an indication of which depths have been thoroughly scanned as opposed to which depths or distances remain to be scanned. One or more buttons 321 may be rendered at various locations of the user interface permitting the operator to initiate a scan of an object and/or manipulate the user interface presented on the display screen 118 or other display in data communication with the scanning device 100. According to one embodiment, the display screen 118 comprises a touch-screen display and the operator may engage button 321 to pause and/or resume an ongoing scan.

Although portion 303a and portion 303b are shown simultaneously in a side-by-side arrangement, other embodiments may be employed without deviating from the scope of the user interface. For example, portion 303a may be rendered in the display screen 118 on the scanning device 100 and portion 303b may be located on a display external to the scanning device 100, and vice versa.

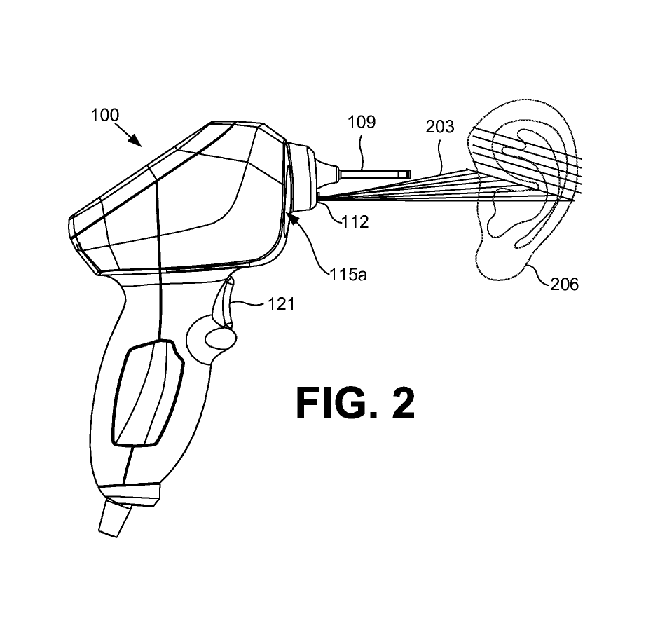

Turning now to FIG. 4, shown is an example drawing of a fiducial marker 403 that may be employed in pose estimation computed during a scan of an ear 206 or other surface. In the non-limiting example of FIG. 4, a fiducial marker 403 may comprise a first circle-of-dots 406a and a second circle-of-dots 406b that generate a ring circumnavigating the fiducial marker 403. Although shown as a circular arrangement, the fiducial marker 403 is not so limited, and may comprise alternatively an oval, square, elliptical, rectangular, or appropriate geometric arrangement.

According to various embodiments of the present disclosure, a circle-of-dots 406 may comprise, for example, a combination of uniformly or variably distributed large dots and a small dots that, when detected, represent a binary number. For example, in the event seven dots in a circle-of-dots 406 are detected in a digital image, the sequence of seven dots may be analyzed to identify (a) the size of the dots and (b) a number or other identifier corresponding to the arrangement of the dots. Detection of a plurality of dots in a digital image may be employed using known region- or blob-detection techniques, as may be appreciated.

As a non-limiting example, a sequence of seven dots comprising small-small-large-small-large-large-large may represent an identifier represented as a binary number of 0-0-1-0-1-1-1 (or, alternatively, 1-1-0-1-0-0-0). The detection of this arrangement of seven dots, represented by the corresponding binary number, may be indicative of a pose of the scanning device 100 relative to the fiducial marker 403. For example, a lookup table may be used to map the binary number to a pose estimate, providing at least an initial estimated pose that may be refined and/or supplemented using information inferred via one or more camera models, as will be discussed in greater detail below. Although the example described above employs a binary operation using a combination of small dots and large dots to form a circle-of-dots 406, variable size dots (having, for example, β sizes) may be employed using variable base numeral systems (for example, a base-β numeral system).

The arrangement of dots in the second circle-of-dots 406b may be the same as the first circle-of-dots 406a, or may vary. If the second circle-of-dots 406b comprises the same arrangement of dots as the first circle-of-dots 406a, then the second circle-of-dots 406b may be used independently or collectively (with the first circle-of-dots 406a) to determine an identifier indicative of the pose of the scanning device 100. Similarly, the second circle-of-dots 406b may be used to determine an error of the pose estimate determined via the first circle-of-dots 406a, or vice versa.

Accordingly, a fiducial marker 403 may be placed relative to the object being scanned to facilitate in accurate pose estimation of the scanning device 100. In the non-limiting example of FIG. 4, the fiducial marker 403 may circumscribe or otherwise surround an ear 206 subject to a scan via the scanning device 100. In one embodiment, the fiducial marker 403 may be detachably attached around the ear of a patient using a headband or similar means.

In other embodiments, a fiducial marker may not be needed, as the tracking targets may be naturally occurring features surrounding and/or within the cavity to be scanned detectable by employing various computer vision techniques. For example, assuming that a person's ear is being scanned by the scanning device 100, the tracking targets may include, hair, folds of the ear, skin tone changes, freckles, moles, and/or any other naturally occurring feature on the person's head relative to the ear.

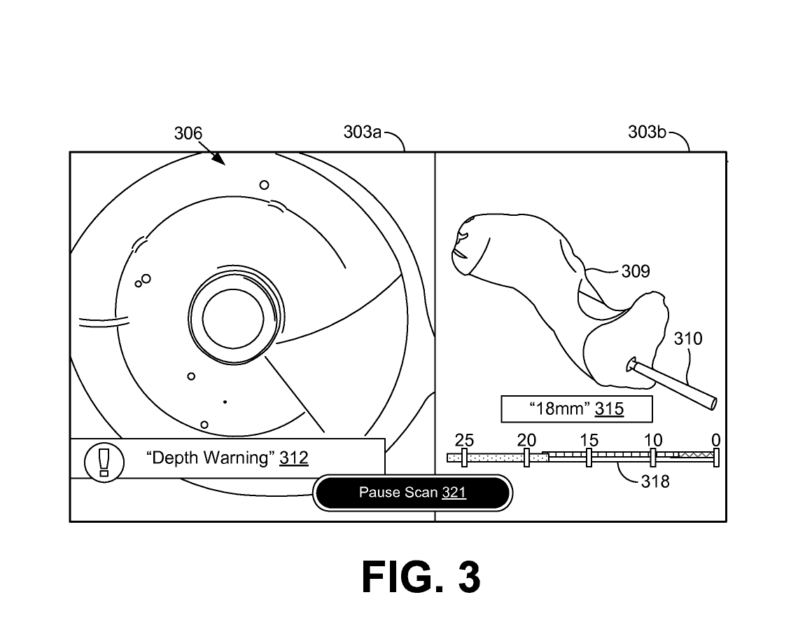

Moving on to FIG. 5, shown is an example of the scanning device 100 conducting a scan of an object. In the non-limiting example of FIG. 5, the scanning device 100 is scanning the surface of an ear 206. However, it should be noted that the scanning device 100 may be configured to scan other types of surfaces and is not limited to human or animal applications. During a scan, a first imaging device 115a and a second imaging device 115b (not shown) may capture digital images of the object subject to the scan. As described above with respect to FIG. 4, a fiducial marker 403 may circumscribe or otherwise surround the object subject to the scan. Thus, while an object is being scanned by the probe 109, the imaging devices 115 may capture images of the fiducial marker 403 that may be used in the determination of a pose of the scanning device 100, as will be discussed in greater detail below.

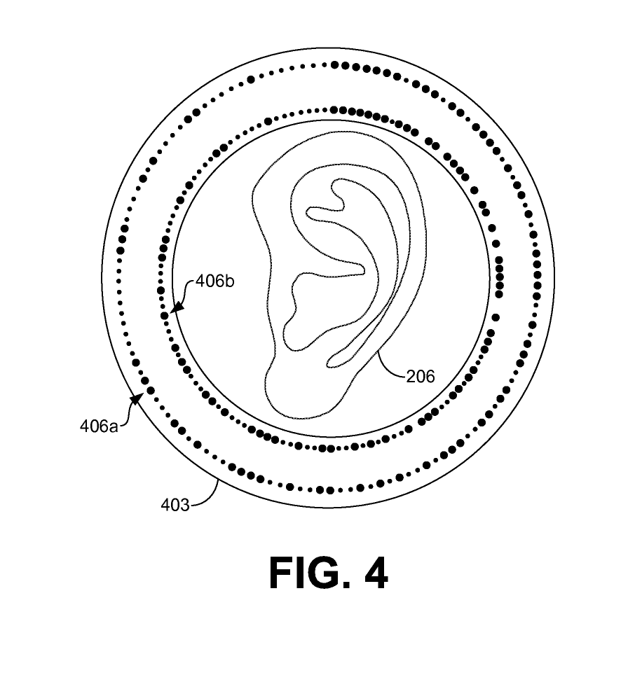

Referring next to FIG. 6, shown is a camera model that may be employed in the determination of world points and image points using one or more digital images captured via the imaging devices 115. Using the camera model of FIG. 6, a mapping between rays and image points may be determined permitting the imaging devices 115 to behave as a position sensor. In order to generate adequate three-dimensional reconstructions of a surface cavity subject to a scan, a pose of a scanning device 100 relative to six degrees of freedom (6DoF) is beneficial.

Initially, a scanning device 100 may be calibrated using the imaging devices 115 to capture calibration images of a calibration object whose geometric properties are known. By employing the camera model of FIG. 6 to the observations identified in the calibration images, internal and external parameters of the imaging devices 115 may be determined. For example, external parameters describe the orientation and position of an imaging device 115 relative to a coordinate frame of an object. Internal parameters describe a projection from a coordinate frame of an imaging device 115 onto image coordinates. Having a fixed position of the imaging devices 115 on the scanning device 100, as depicted in FIGS. 1A-1C, permits the determination of the external parameters of the scanning device 100 as well. The external parameters of the scanning device 100 may be used to generate three-dimensional reconstructions of a surface cavity subject to a scan.

In the camera model of FIG. 6, projection rays meet at a camera center defined as C, wherein a coordinate system of the camera may be defined as X_{c}, Y_{c}, Z_{c}, where Z_{c} is defined as the principal axis 603. A focal length f defines a distance from the camera center to an image plane 606 of an image captured via an imaging device 115. Using a calibrated camera model, perspective projections may be represented via:

$$( \begin{matrix}x \\ y \\ 1\end{matrix} ) ≃ [ \begin{matrix}f & 0 & 0 & 0 \\ 0 & f & 0 & 0 \\ 0 & 0 & 1 & 0\end{matrix} ] ( \begin{matrix}{X}_{c} \\ {Y}_{c} \\ {Z}_{c} \\ 1\end{matrix} ) \qquad ( eq . 1 )$$

A world coordinate system 609 with principal point O may be defined separately from the camera coordinate system as X_{O}, Y_{O}, Z_{O}. According to various embodiments, the world coordinate system 609 may be defined at a base location of the probe 109 of the scanning device 100, however, it is understood that various locations of the scanning device 100 may be used as the base of the world coordinate system 609. Motion between the camera coordinate system and the world coordinate system 609 is defined by a rotation R, a translation t, a tilt φ. A principal point p is defined as the origin of a normalized image coordinate system (x, y) and a pixel image coordinate system is defined as (u, v), wherein α is

$$( \frac{π}{2} )$$

in a conventional orthogonal pixel coordinate axes. The mapping of a three-dimensional point X to the digital image m is represented via:

$$\begin{matrix}m ≃ [ \begin{matrix}{m}_{u} & - {m}_{u} cot ( α ) & {u}_{0} \\ 0 & \frac{{m}_{v}}{sin ( α )} & {v}_{0} \\ 0 & 0 & 1\end{matrix} ] [ \begin{matrix}f & 0 & 0 & 0 \\ 0 & f & 0 & 0 \\ 0 & 0 & 1 & 0\end{matrix} ] [ \begin{matrix}R & t \\ 0 & 1\end{matrix} ] X \\ = [ \begin{matrix}{m}_{u} f & - {m}_{u} f cot ( α ) & {u}_{0} \\ 0 & \frac{{m}_{v}}{sin ( α )} f & {v}_{0} \\ 0 & 0 & 1\end{matrix} ] [ \begin{matrix}R & t\end{matrix} ] X\end{matrix} \qquad ( eq . 2 )$$

Further, the camera model of FIG. 6 may account for distortion deviating from a rectilinear projection. Radial distortion generated by various lenses of an imaging device 115 may be incorporated into the camera model of FIG. 6 by considering projections in a generic model represented by:

r(θ)=1+k₂θ³+k₃θ⁵+k₄θ⁷+ . . . (eq. 3)

As eq. 3 shows a polynomial with four terms up to the seventh power of θ, the polynomial of eq. 3 provides enough degrees of freedom (e.g., six degrees of freedom) for a relatively accurate representation of various projection curves that may be produced by a lens of an imaging device 115. Other polynomial equations with lower or higher orders or other combinations of orders may be used.

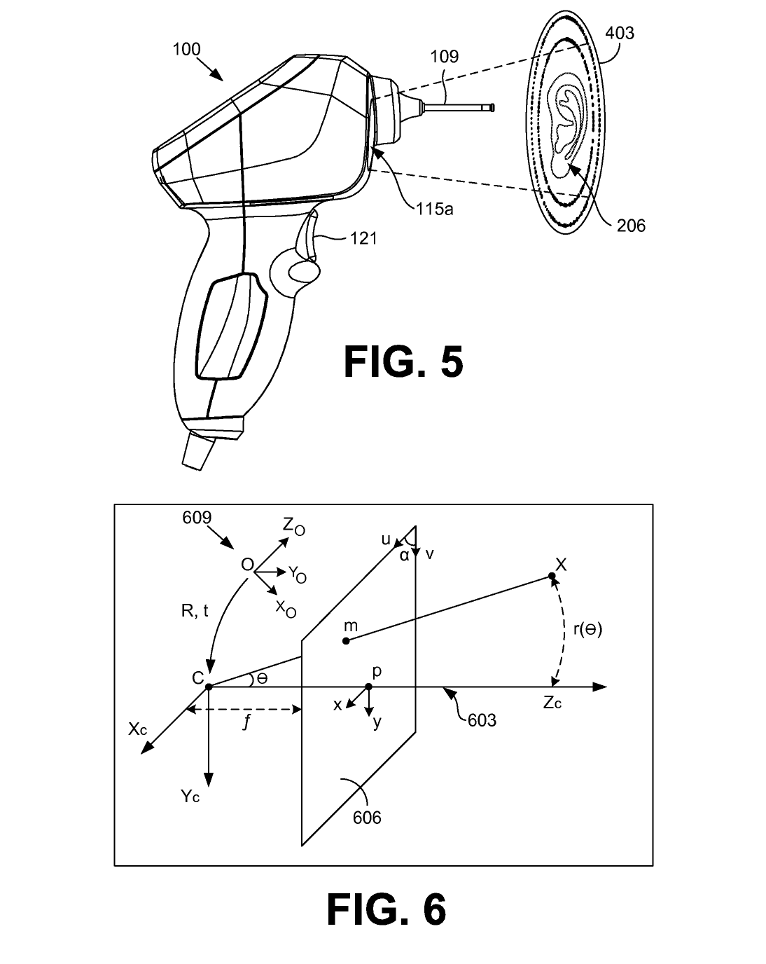

Turning now to FIG. 7, shown is another drawing of a portion of the scanning device 100 according to various embodiments. In this example, the scanning device 100 comprises a first imaging device 115a and a second imaging device 115b, all implemented in a fashion similar to that of the scanning device described above with reference to FIGS. 1A-1C. The first imaging device 115a and the second imaging device 115b may be mounted within the body 103 without hindering or impeding a view of the first imaging device 115a and/or the second imaging device 115b.

The placement of two imaging devices 115 permits computations of positions using epipolar geometry. For example, when the first imaging device 115a and the second imaging device 115b view a three-dimensional scene from their respective positions (different from the other imaging device 115), there are geometric relations between the three-dimensional points and their projections on two-dimensional images that lead to constraints between the image points. These geometric relations may be modeled via the camera model of FIG. 6 and may incorporate the world coordinate system 609 and one or more camera coordinate systems (e.g., camera coordinate system 703a and camera coordinate system 703b).

By determining the internal parameters and external parameters for each imaging device 115 via the camera model of FIG. 6, the camera coordinate system 703 for each of the imaging devices 115 may be determined relative to the world coordinate system 609. The geometric relations between the imaging devices 115 and the scanning device 100 may be modeled using tensor transformation (e.g., covariant transformation) that may be employed to relate one coordinate system to another. Accordingly, a device coordinate system 706 may be determined relative to the world coordinate system 609 using at least the camera coordinate systems 703a-b. As may be appreciated, the device coordinate system 706 relative to the world coordinate system 609 comprises the pose estimate of the scanning device 100.

In addition, the placement of the two imaging device 115 in the scanning device 100 may be beneficial in implementing computer stereo vision. For example, both imaging devices 115 can capture digital images of the same scene; however, they are separated by a distance 709. A processor in data communication with the imaging devices 115 may compare the images by shifting the two images together over the top of each other to find the portions that match to generate a disparity used to calculate a distance between the scanning device 100 and the object of the picture. However, implementing the camera model of FIG. 6 is not as limited as an overlap between two digital images taken by a respective imaging device 115 is not warranted when determining independent camera models for each imaging device 115.

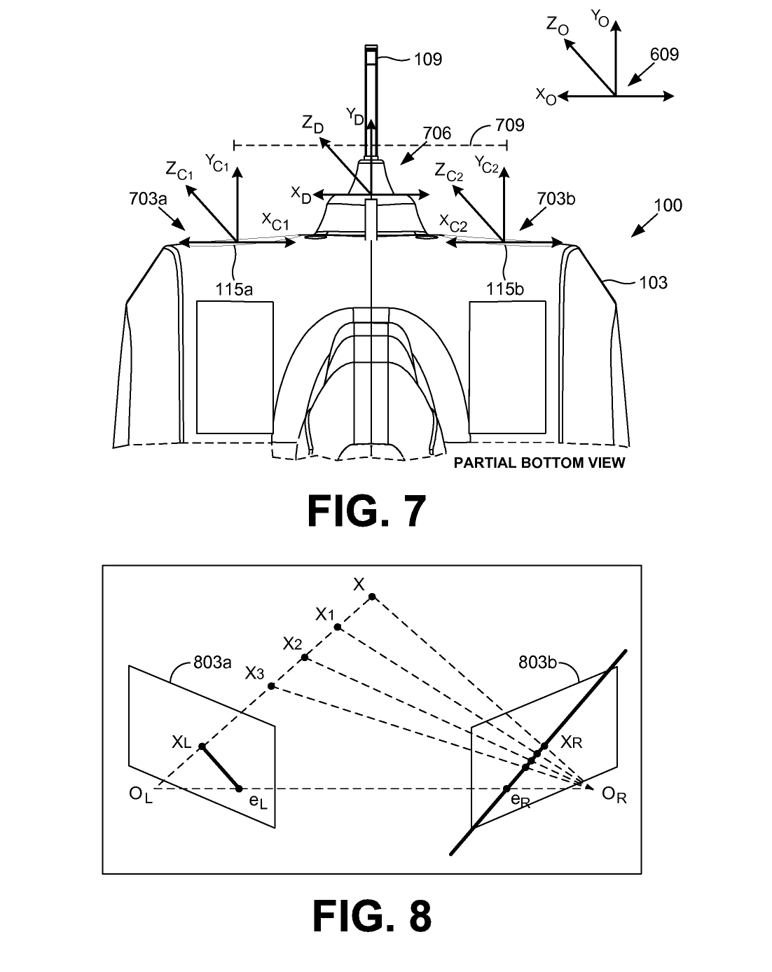

Moving on to FIG. 8, shown is the relationship between a first image 803a captured, for example, by the first imaging device 115a and a second image 803b, for example, captured by the second imaging device 115b. As may be appreciated, each imaging device 115 is configured to capture a two-dimensional image of a three-dimensional world. The conversion of the three-dimensional world to a two-dimensional representation is known as perspective projection, which may be modeled as described above with respect to FIG. 6. The point X_{L} and the point X_{R} are shown as projections of point X onto the image planes. Epipole e_{L} and epipole e_{R} have centers of projection O_{L} and O_{R} on a single three-dimensional line. Using projective reconstruction, the constraints shown in FIG. 8 may be computed.

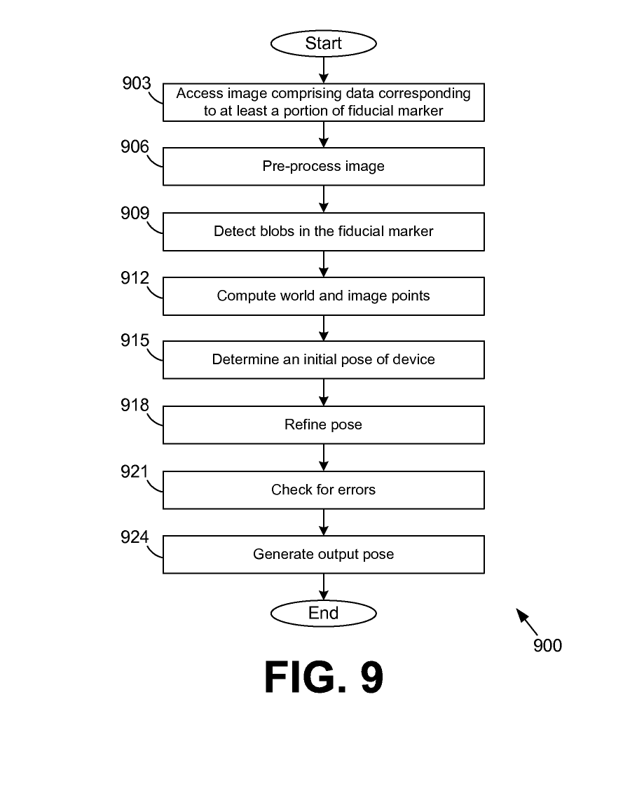

Referring next to FIG. 9, shown is a flowchart that provides one example of the operation of a portion of a pose estimate application 900 that may be executed by a processor, circuitry, and/or logic according to various embodiments. It is understood that the flowchart of FIG. 9 provides merely an example of the many different types of functional arrangements that may be employed to implement the operation of the portion of the pose estimate application 900 as described herein. As an alternative, the flowchart of FIG. 9 may be viewed as depicting an example of elements of a method implemented in a processor in data communication with a scanning device 100 (FIGS. 1A-1C) according to one or more embodiments.

Beginning with 903, a digital image comprising data corresponding to at least a portion of fiducial marker 403 (FIG. 4) may be accessed. A digital image may have been generated, for example, via the one or more imaging devices 115 (FIGS. 1A-1C) in data communication with the scanning device 100. As may be appreciated, a digital image may comprise a finite number of pixels representing a two-dimensional image according to a resolution capability of the imaging device 115 employed in the capture of the digital image. As will be discussed in 909, the pixels may be analyzed using region- or blob-detection techniques to identify: (a) the presence of a fiducial marker 403 in the digital image; and (b) if the fiducial marker 403 is present in the digital image, identify dots in a first circle-of-dots 406a (FIG. 4) and/or a second circle-of-dots 406b (FIG. 4) (or other arrangement), as depicted in FIG. 4.

As the digital image will be analyzed using one or more region- or blob-detection techniques, it may be beneficial to prepare a digital image for blob-detection. In 906, the digital image accessed in 903 may be pre-processed according to predefined parameters (e.g., internal and external parameters, discussed above). Pre-processing a digital image according to predefined parameters may comprise, for example, applying filters and/or modifying chroma, luminescence, and/or other features of the digital image. In addition, pre-processing may further comprise, for example, removing speckles or extraneous artifacts from the digital image, removing partial dots from the digital image, etc.

As discussed above, in 909, blob detection may be employed to identify: (a) the presence of a fiducial marker in the digital image; and (b) if the fiducial marker is present in the digital image, identify dots in a circle-of-dots 406 (or other arrangement), as depicted in FIG. 4. As a non-limiting example, blob-detection may comprise detecting regions in the digital image that differ in properties according to respective pixel values. Such properties may comprise brightness (also known or luminescence) or color. Thus, when a representative pixel or region of pixels is brighter and/or of a different color than a surrounding pixel or region of pixels, a region or blob in the digital image may be identified. The detection of circles in a circle-of-dots 406 may present a sequence of circles that are indicative of a position of the scanning device 100 relative to the fiducial marker 403, as well as the object being scanned.

For example, a sequence of seven dots comprising small-small-large-small-large-large-large may represent a binary number of 0-0-1-0-1-1-1 (or, alternatively, 1-1-0-1-0-0-0). The detection of this sequence of seven dots, represented by the binary number, is indicative of a pose of the scanning device 100 relative to the fiducial marker 403. According to one embodiment, a lookup table may be used to map the binary number to a pose estimate, providing at least an initial pose estimate that may be refined and/or supplemented using information inferred via one or more camera models, as will be discussed in 912. According to various embodiments, the initial pose estimate may provide enough information to determine six degrees of freedom of the scanning device 100. As more dots are identified, a more approximate identifier may be determined indicating a more approximate pose estimate of the scanning device 100.

Next, in 912, world and image points may be computed to refine and/or supplement the information determined from the fiducial marker 403. According to one embodiment, the camera model of FIG. 6 may be employed to determine geometric measurements from the digital image. As discussed above with respect to FIG. 6, the camera model comprises both external parameters and internal parameters that may be determined during a calibration of the scanning device 100 and/or the imaging devices 115 in data communication with the scanning device. External parameters describe the camera orientation and position to a coordinate from of an object. Internal parameters describe a projection from the camera coordinate frame onto image coordinates. The parameters may be determined via the camera model of FIG. 6 and may be used to refine and/or supplement the data determined from the fiducial marker 403.

In 915, the world and image points may be used in an initial pose of the scanning device 100 (i.e., the pose estimate). For example, an identifier determined from at least a portion of an identifier identified in a digital image may be indicative of a pose estimate of the scanning device. Similarly, after a determination of the external parameters and internal parameters for one or more imaging devices 115 has been determined via a camera model, a pose estimate of the scanning device 100 may be determined relative to a world coordinate system 609 (FIGS. 6 and 7). According to various embodiments, the device coordinate system 706 may be positioned at the base of the probe 109 (FIGS. 1A-1C and FIG. 7). Determining a pose of the scanning device 100 relative to six degrees of freedom in a world coordinate system 609 may be sufficient for an accurate pose output.

In 918, the pose estimate may be refined. For example, a second digital image of the fiducial marker 403 comprising one or more circle-of-dots 406 captured via the imaging devices 115, if detected, may be used in refining and/or error checking the computed pose estimate, as shown in 921. In 924, an output of the pose of the scanning device 100 may be transmitted and/or accessed by other components in data communication with the scanning device 100. For example, the pose estimate may be requested from a requesting service such as a service configured to generate a three-dimensional reconstruction of an object being scanned using the scanning device 100. The pose estimate may provide information beneficial in the three-dimensional reconstruction of the object, such as the distance of the scanning device 100 relative to a surface cavity being scanned by the scanning device 100.

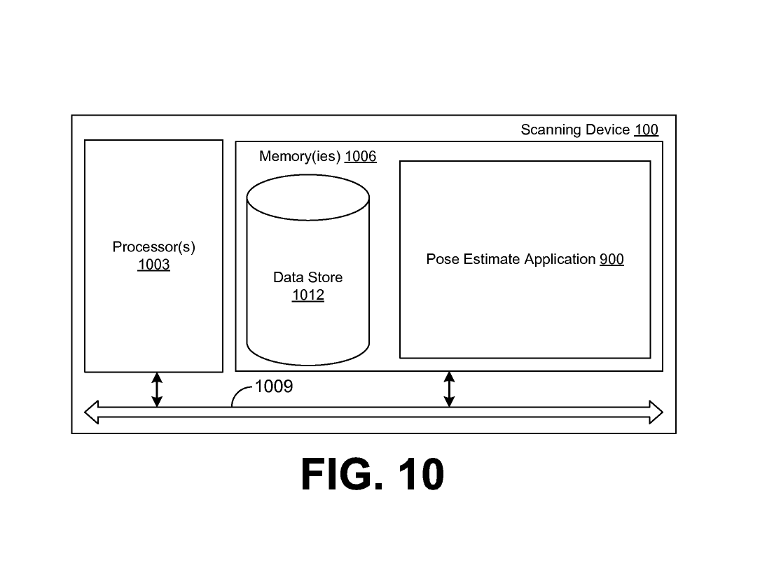

With reference to FIG. 10, shown is a schematic block diagram of a scanning device 100 according to an embodiment of the present disclosure. A scanning device 100 may comprise at least one processor circuit, for example, having a processor 1003 and a memory 1006, both of which are coupled to a local interface 1009. The local interface 1009 may comprise, for example, a data bus with an accompanying address/control bus or other bus structure as can be appreciated.

Stored in the memory 1006 are both data and several components that are executable by the processor 1003. In particular, a pose estimate application 900 is stored in the memory 1006 and executable by the processor 1003, as well as other applications. Also stored in the memory 1006 may be a data store 1012 and other data. In addition, an operating system may be stored in the memory 1006 and executable by the processor 1003.

It is understood that there may be other applications that are stored in the memory 1006 and are executable by the processor 1003 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 1006 and are executable by the processor 1003. In this respect, the term “executable” means a program file that is in a form that can ultimately be run by the processor 1003. Examples of executable programs may be, for example, a compiled program that can be translated into machine code in a format that can be loaded into a random access portion of the memory 1006 and run by the processor 1003, source code that may be expressed in proper format such as object code that is capable of being loaded into a random access portion of the memory 1006 and executed by the processor 1003, or source code that may be interpreted by another executable program to generate instructions in a random access portion of the memory 1006 to be executed by the processor 1003, etc. An executable program may be stored in any portion or component of the memory 1006 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1006 is defined herein as including both volatile and nonvolatile memory and data storage components. Volatile components are those that do not retain data values upon loss of power. Nonvolatile components are those that retain data upon a loss of power. Thus, the memory 1006 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores and the memory 1006 may represent multiple memories 1006 that operate in parallel processing circuits, respectively. In such a case, the local interface 1009 may be an appropriate network that facilitates communication between any two of the multiple processors 1003, between any processor 1003 and any of the memories 1006, or between any two of the memories 1006, etc. The local interface 1009 may comprise additional systems designed to coordinate this communication, including, for example, performing load balancing. The processor 1003 may be of electrical or of some other available construction.

Although the pose estimate application 900, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowchart of FIG. 9 shows the functionality and operation of an implementation of portions of the pose estimate application 900. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor 1003 in a computer system or other system. The machine code may be converted from the source code, etc. If embodied in hardware, each block may represent a circuit or a number of interconnected circuits to implement the specified logical function(s).

Although the flowchart of FIG. 9 shows a specific order of execution, it is understood that the order of execution may differ from that which is depicted. For example, the order of execution of two or more blocks may be scrambled relative to the order shown. Also, two or more blocks shown in succession in FIG. 9 may be executed concurrently or with partial concurrence. Further, in some embodiments, one or more of the blocks shown in FIG. 9 may be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein, including the pose estimate application 900, that comprises software or code can be embodied in any non-transitory computer-readable medium for use by or in connection with an instruction execution system such as, for example, a processor 1003 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the pose estimate application 900, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same scanning device 100, or in multiple computing devices in a common computing environment. Additionally, it is understood that terms such as “application,” “service,” “system,” “engine,” “module,” and so on may be interchangeable and are not intended to be limiting.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A system, comprising:
a mobile computing device capable of data communication with an imaging device configured to conduct a scan of an object; and
a pose estimate application executable in the mobile computing device, the pose estimate application comprising logic that:
analyzes a digital image captured via the imaging device to determine a plurality of parameters associated with the imaging device according to at least one camera model;
determines a position of the imaging device relative to a world coordinate system utilizing at least the plurality of parameters; and
approximates a pose of the mobile device in a three-dimensional space relative to the object subject to the scan utilizing at least the plurality of parameters.

2. The system of claim 1, wherein the at least one camera model further comprises a lens distortion model accounting for distortion in the digital image produced by a lens of the imaging device.

3. The system of claim 1, wherein the pose estimate application further comprises logic that:
analyzes the digital image to identify a plurality of regions in a fiducial marker captured within the digital image, the digital image comprising pixel data corresponding to at least a portion of the fiducial marker;
determines a respective size for individual ones of the plurality of regions identified within the fiducial marker;
generates an identifier indicative of the pose of the mobile computing device based at least in part on an arrangement of sizes of the plurality of regions within the fiducial marker; and
refines the pose of the mobile computing device in the three-dimensional space utilizing at least the identifier.

4. The system of claim 3, wherein the fiducial marker further comprises a circle-of-dots pattern.

5. The system of claim 4, wherein the circle-of-dots pattern further comprises at least a first circle-of-dots pattern and a second circle-of-dots pattern.

6. The system of claim 1, wherein the pose estimate application further comprises logic that outputs the pose of the mobile computing device to a requesting service to generate a three-dimensional reconstruction of the object using at least the pose of the mobile computing device in the three-dimensional space.

7. The system of claim 1, wherein the mobile computing device further comprises an otoscanner configurable to scan an ear canal.

8. A method, comprising:
analyzing, by a processor in data communication with a scanning device comprising at least one imaging device, a digital image captured via the at least one imaging device to determine a plurality of parameters associated with the at least one imaging device according to at least one camera model;
determining, by the processor, a position of the imaging device relative to a world coordinate system utilizing at least the plurality of parameters; and
approximating, by the processor, a pose of the scanning device in a three-dimensional space relative to an object subject to a scan utilizing at least the plurality of parameters.

9. The method of claim 8, wherein the at least one camera model further comprises a lens distortion model accounting for distortion in the digital image produced by a lens of the imaging device.

10. The method of claim 8, further comprising:
analyzing, by the processor, a digital image to identify a plurality of regions in a fiducial marker captured within the digital image, the digital image comprising pixel data corresponding to at least a portion of the fiducial marker;
determining, by the processor, a respective size for individual ones of the plurality of regions identified within the fiducial marker;
generating, by the processor, an identifier indicative of the pose of the scanning device based at least in part on an arrangement of sizes of the plurality of regions within the fiducial marker; and
refining, by the processor, the pose of the scanning device in the three-dimensional space utilizing at least the identifier.

11. The method of claim 10, wherein the fiducial marker further comprises a circle-of-dots pattern.

12. The method of claim 11, wherein the circle-of-dots pattern further comprises at least a first circle-of-dots pattern and a second circle-of-dots pattern.

13. The method of claim 8, further comprising generating, by the processor, the pose of the scanning device to a requesting service to generate a three-dimensional reconstruction of the object using at least the pose of the scanning device in the three-dimensional space.

14. The method of claim 8, wherein the scanning device further comprises an otoscanner configurable to scan an ear canal.

15. A non-transitory computer-readable medium embodying a program executable in at least one otoscanner configurable to scan a cavity, the program comprising code that:
analyzes a digital image captured via an imaging device communicable with the at least one otoscanner to determine a plurality of parameters associated with the imaging device according to at least one camera model;
determines a position of the imaging device relative to a world coordinate system utilizing at least the plurality of parameters; and
approximates a pose of the at least one otoscanner in a three-dimensional space relative to the cavity subject to the scan utilizing at least the plurality of parameters; and
transmits the pose of the otoscanner to a requesting service to generate a three-dimensional reconstruction of the cavity using at least the pose of the otoscanner in the three-dimensional space.

16. The non-transitory computer-readable medium of claim 15, wherein the at least one camera model further comprises a lens distortion model accounting for distortion in the digital image produced by a lens of the imaging device.

17. The non-transitory computer-readable medium of claim 15, the program further comprising code that:
analyzes the digital image to identify a plurality of regions in a fiducial marker captured within the digital image, the digital image comprising pixel data corresponding to at least a portion of the fiducial marker;
determines a respective size for individual ones of the plurality of regions identified within the fiducial marker;
generates an identifier indicative of the pose of the otoscanner based at least in part on an arrangement of sizes of the plurality of regions within the fiducial marker; and
refines the pose of the otoscanner in the three-dimensional space utilizing at least the identifier.

18. The non-transitory computer-readable medium of claim 17, wherein the fiducial marker further comprises a circle-of-dots pattern.

19. The non-transitory computer-readable medium of claim 18, wherein the circle-of-dots pattern further comprises at least a first circle-of-dots pattern and a second circle-of-dots pattern.

20. The non-transitory computer-readable medium of claim 15, wherein the cavity further comprises an ear canal.
